(19) United States
(12) Patent Application Publication
(10) Pub. No.: US 2004/0143317 A1
(43) Pub. Date: Jul. 22, 2004

(54) Medical devices

(76) Inventors: Jonathan S. Stinson, Minneapolis, MN (US); Robert A. Vanderlaan, Maple Grove, MN (US)

Correspondence Address: FISH & RICHARDSON PC, 225 FRANKLIN ST, BOSTON, MA, 02110, US

(21) Appl. No.: 10/346,487

(22) Filed: Jan. 17, 2003

(51) Int. Cl. A61F 2/06

(52) U.S. Cl. 623/1.15; 623/1.44

(57) ABSTRACT

Medical devices, such as stents, stent-grafts, grafts, guidewires, and filters, having enhanced radiopacity are disclosed.

### TECHNICAL FIELD

The invention relates to medical devices, such as, for example, stents, stent-grafts, guidewire, and filters, and methods of making the devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprosthesis include stents and covered stents, sometimes called “stent-grafts”.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially.

For example, the expansion mechanism can include the catheter carrying a balloon, which carries a balloon-expandable endoprosthesis. The balloon can be inflated to deform and to fix the expanded endoprosthesis at a predetermined position in contact with the lumen wall.

The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force. Alternately, self-expansion can occur through a material phase transition, induced by a change in temperature or by application of a stress.

To support a passageway open, endoprostheses are sometimes made of relatively strong materials, such as stainless steel or Nitinol (a nickel-titanium alloy), formed into struts or wires. These materials, however, can be relatively radiolucent. That is, the materials may not be easily visible under X-ray fluoroscopy, which is a technique used to locate and to monitor the endoprostheses during and after delivery. To enhance their visibility (e.g., by increasing their radiopacity), the endoprostheses can be coated with a relatively radiopaque material, such as gold, and/or include one or more radiopaque markers.

### SUMMARY

The invention relates to medical devices.

In one aspect, the invention features a medical device, such as an endoprosthesis, having a first portion that is radiopaque and mechanically relatively weak, and a second portion that is less radiopaque than the first portion. The second portion, e.g., made of a superelastic, shape memory material, is capable of providing the device with strength, e.g., to support open a body vessel. The first portion is capable of enhancing the radiopacity of the device without inhibiting the performance of the second portion.

In another aspect, the invention features a stent including a structure having a first portion including a first composition, the first composition fracturing upon expansion of the structure, and a second portion including a second composition less radiopaque than the first composition.

The second portion can surround the first portion.

The second composition can include a shape memory material and/or has superelastic characteristics: The second composition can include a nickel-titanium alloy, stainless steel, titanium, and/or a polymer. The polymer can be, for example, polynorbornene, polycaprolactone, polyenes, nylons, polycyclooctene (PCO), or polyvinyl acetate/polyvinylidinefluoride.

The first composition can have a density greater than about 9.9 g/cc. The first composition can include gold, tantalum, palladium, and/or platinum. The first composition can be in the form of a powder and/or in the form of fibers.

The structure can include a third portion having the second composition, and the first portion is between the second and third portions.

The structure can be in the form of a wire or a tubular member.

The stent can be a self-expandable stent, a balloon-expandable stent, or a stent-graft, e.g., including a therapeutic agent.

In another aspect, the invention features a medical device including a structure including a first portion having a mixture including a radiopaque composition and a second composition, the mixture having a yield strength less than a yield strength of the substantially pure radiopaque composition, and a second portion having a third composition less radiopaque than the mixture.

Embodiments may include one or more of the following features. The second composition includes carbon, nitrogen, hydrogen, calcium, potassium, bismuth, and/or oxygen. The first portion has a yield strength less than about 80 ksi. The third composition includes a shape memory material and/or has superelastic characteristics. The third composition includes a nickel-titanium alloy, a stainless steel, or a shape memory polymer. The first composition has a density greater than about 9.9 g/cc. The first composition includes gold, tantalum, palladium, and/or platinum. The first composition is in the form of a powder. The first composition is in the form of fibers. The structure further includes a third portion having the third composition, and the first portion is between the second and third portions.

The structure can be in the form of a wire or a tubular member. The device can be a self-expandable stent, a balloon-expandable stent, a stent-graft, e.g., including a therapeutic agent, or an intravascular filter.

In another aspect, the invention features a method of making a medical device. The method includes reducing a yield strength of a radiopaque composition, and incorporating the radiopaque composition into the medical device.

Embodiments may include one or more of the following features. Reducing the yield strength includes annealing the radiopaque composition. Reducing the yield strength includes reacting the radiopaque composition with a second composition include carbon, nitrogen, hydrogen, calcium, potassium, bismuth, and/or oxygen. Reducing the yield strength includes removing selected portions of the radiopaque composition. The yield strength of radiopaque composition is reduced to less than about 80 ksi.

In another aspect, the invention features a method of making a medical device, including forming a structure having a first portion including a first composition, and a second portion including a second composition less radiopaque than the first composition; incorporating the structure into the medical device; and reducing a yield strength of the first composition.

Embodiments may include one or more of the following features. Reducing the yield strength is performed after incorporating the structure into the medical device. Reducing the yield strength includes reacting the first composition with a third composition. Reducing the yield strength includes heating the first composition. The structure is in the form of a wire. The structure is in the form of a tube.

In another aspect, the invention features a method of making a medical device, including forming a structure having a first portion including a first composition, and a second portion including a second composition less radiopaque than the first composition; and incorporating the structure into the medical device, the first composition weakening in response to the incorporating of the structure.

Embodiments may include one or more of the following features. The medical device includes a stent delivery system. The method further includes forming the structure into an endoprosthesis.

In another aspect, the invention features a medical device including a structure including a first portion having a first composition, the first composition weakening upon deformation of the structure, and a second portion having a second composition less radiopaque than the first composition. For example, during deformation of the structure, such as during expansion, the first composition can be deformed beyond its plastic limit so as to separate, e.g., fracture or crack, and to provide numerous discontinuities in the first portion. The discontinuities can be detected, for example, using X-ray techniques. In some cases, the first composition is not expected to flow with the second composition upon deformation of the structure.

The second portion can surround the first portion.

The second composition can include a shape memory material and/or has superelastic characteristics. The second composition can include a nickel-titanium alloy, stainless steel, titanium, and/or a polymer. The polymer can be, for example, polynorbornene, polycaprolactone, polyenes, nylons, polycyclooctene (PCO), or polyvinyl acetate/polyvinylidinefluoride.

The first composition can have a density greater than about 9.9 g/cc. The first composition can include gold, tantalum, palladium, and/or platinum. The first composition can be in the form of a powder and/or in the form of fibers.

The structure can include a third portion having the second composition, and the first portion is between the second and third portions.

The structure can be in the form of a wire or a tubular member.

The device can be a self-expandable stent, a balloon-expandable stent, a stent-graft, e.g., including a therapeutic agent, or an intravascular filter.

In certain embodiments, the structure, e.g., in the form of a wire, can be used to form guidewires, filters, filter wires, catheter reinforcement wires, snares, embolic coils, leadwires, e.g., for pacemakers, clips, or other devices in which it is desirable to have enhanced radiopacity with the use of elastic or shape memory deformable/recoverable materials.

Other aspects, features, and advantages of the invention will be apparent from the description of the preferred embodiments thereof and from the claims.

### DESCRIPTION OF DRAWINGS

FIG. 1 is a perspective view of an embodiment of an endoprosthesis.

FIG. 2A is a cross-sectional view of an embodiment of a wire; and FIG. 2B is a cross-sectional view of the wire of FIG. 2A, taken along line 2B-2B.

FIG. 3 is a cross-sectional view of an embodiment of a wire.

FIG. 4 illustrates an embodiment of a method of making an endoprosthesis.

### DETAILED DESCRIPTION

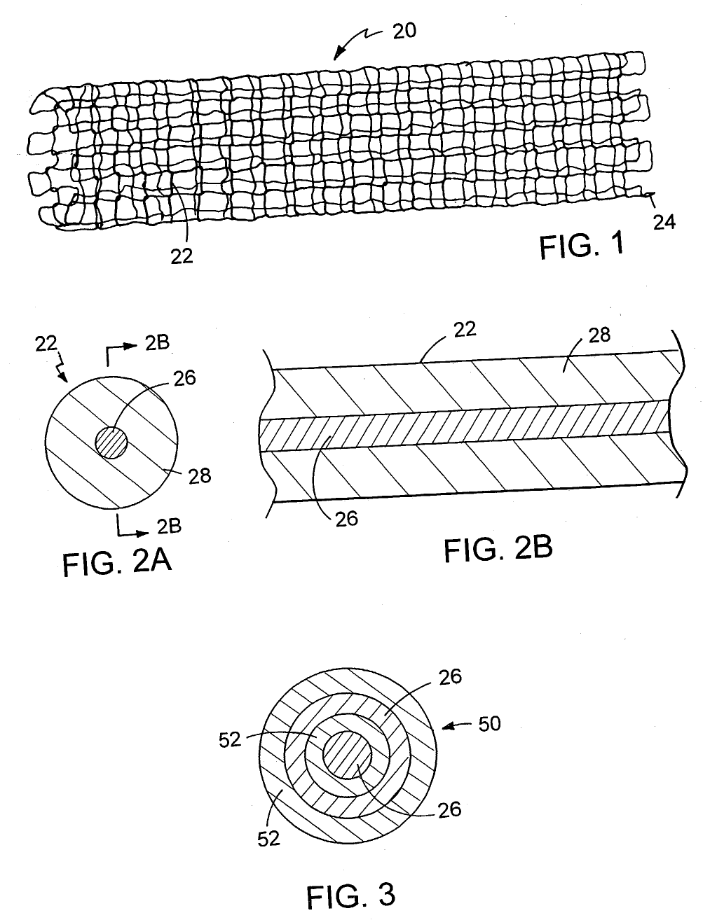

Referring to FIGS. 1, 2A, and 2B, an endoprosthesis 20 (as shown, a self-expandable stent) includes a filament or wire 22 formed, e.g., knitted, into a tubular member 24. Wire 22 includes a composite structure formed of a relatively radiopaque portion 26 concentrically surrounded by an outer portion 28. Outer portion 28 is capable of providing endoprosthesis 20 with desirable mechanical properties (such as high elasticity and strength) and chemical properties (such as biocompatibility). As described below, radiopaque portion 26 can be formed of one or more materials selected and/or designed to be mechanically weak relative to forces exerted by endoprosthesis 20 during use, e.g., expansion. As a result, radiopaque portion 26 is capable of enhancing the radiopacity of endoprosthesis 20, while not substantially affecting, e.g., inhibiting, the performance of outer portion 28 and the endoprosthesis.

Radiopaque portion 26 can include one or more radiopaque materials, e.g., a metal or a mixture of metals. In certain embodiments, the radiopaque material is relatively absorptive of X-rays, e.g., having a linear attenuation coefficient of at least 25 cm⁻¹, e.g., at least 50 cm⁻¹, at 100 keV. In some embodiments, the radiopaque material is relatively dense to enhance radiopacity, e.g., having a density of about 9.9 g/cc or greater. For example, the radiopaque material can include tantalum (16.6 g/cc), tungsten (19.3 g/cc), rhenium (21.2 g/cc), bismuth (9.9 g/cc), silver (16.49 g/cc), gold (19.3 g/cc), platinum (21.45 g/cc), iridium (22.4 g/cc), and/or their alloys.

Radiopaque portion 26 is formed and/or is modified such that the performance of outer portion 28 and endoprosthesis 20 is not adversely affected. In certain embodiments, radiopaque portion 26 can be formed to have a yield strength less than forces exerted by endoprosthesis 20 during use. For example, for a Nitinol stent, radiopaque portion 26 can have a yield strength less than a recovery stress of about 80 ksi exerted by the Nitinol. Alternatively or in addition, radiopaque portion 26 can be designed to mechanically weaken or fail, e.g., fracture, crack, deform, or disintegrate, as endoprosthesis 20 is used. Numerous methods of forming or modifying radiopaque portion 26 are possible.

In some embodiments, the radiopaque material can be selectably heat treated, e.g., annealed, to weaken or to soften the material. Generally, the radiopaque material is heat treated to provide a yield stress less than a recovery stress of outer portion 28 and/or endoprosthesis 20. An example of heat treating the radiopaque material is provided below in Example 1.

In some embodiments, the radiopaque material can be made relatively weak or brittle by reacting the material with another material(s). For example, tantalum can be embrittled by introducing small amounts of impurities, such as carbon, oxygen, nitrogen, and/or hydrogen. The impurities can be introduced by heating, e.g., annealing, the tantalum in an atmosphere containing air, nitrogen, nitrogen-hydrogen, and/or carbon dioxide. The embrittled tantalum can fracture into smaller particles, e.g., during processing operations, such as rolling or drawing, described below. Gold can be embrittled by heating in a bath containing ions of bismuth, calcium, or potassium, and allowing the ions to diffuse into the gold. For a Nitinol/gold composite wire, the embrittlement of gold can be performed concurrently with the annealing of Nitinol. For example, the wire can be formed such that selected portions of gold are exposed, e.g., by removing or grinding portions of Nitinol, and the wire can then be heat treated in a fluidized bed or a heated salt bath.

In some embodiments, the radiopaque material can be in a form that in aggregate makes radiopaque portion 26 relatively weak, e.g., susceptible to fracturing or cracking. The radiopaque material can be in the form of a powder, particulates, shards, and/or fibers, such that radiopaque portion 26 is not a continuously solid core.

The fibers can be generally elongated structures having lengths greater than widths or diameters. The fibers can have a length of about 0.1 mm to about 10 mm. In some embodiments, the fibers can have a length equal to or greater than about 0.1, 0.5, 1.0, 1.5, 2.0, 2.5, 3.0, 3.5, 4.0, 4.5, 5.0, 5.5, 6.0, 7.0, 7.5, 8.0, 8.5, 9.0, or 9.5 mm; and/or equal to or less than about 10, 9.5, 9.0, 8.5, 8.0, 7.5, 7.0, 6.5, 6.0, 5.5, 5.0, 4.5, 4.0, 3.5, 3.0, 2.5, 2.0, 1.5, 1.0, or 0.5 mm, e.g., about 0.1 to about 3.0 mm. The lengths of the fibers may be uniform or relatively random. The fibers can have a width of about 1 micron to about 100 microns. The fibers can have a width equal to or greater than about 1, 10, 20, 30, 40, 50, 60, 70, 80, or 90 microns; and/or equal to or less than about 100, 90, 80, 70, 60, 50, 40, 30, 20, or 10 microns, e.g., about 1 to about 20 microns. The widths can be uniform or relatively random.

In some embodiments, the fibers have length to width aspect ratios from about 10:1 to about 100:1, although higher aspect ratios are possible. In some embodiments, the length to width aspect ratios can be equal to or greater than about 10:1, 20:1, 30:1, 40:1, 50:1, 60:1, 70:1, 80:1, or 90:1; and/or equal to or less than about 100:1, 90:1, 80:1, 70:1, 60:1, 50:1, 40:1, 30:1, or 20:1, e.g., about 20:1 to about 40:1. The width used to determine the aspect ratio can be the narrowest or broadest width. The length can be the largest dimension of a fiber. Mixtures of fibers having two or more different aspect ratios and/or dimensions can be used.

The fibers can have a variety of configurations or shapes. The fibers can have a cross section that is circular or non-circular, such as oval, or regularly or irregularly polygonal having 3, 4, 5, 6, 7, or 8 or more sides. The outer surface of the fibers can be relatively smooth, e.g., cylindrical or rod-like, or faceted. The fibers can have uniform or non-uniform thickness, e.g., the fibers can taper along their lengths. Mixtures of fibers having two or more different configurations or shapes can be used. In other embodiments, thin, flat shard-like fibers having irregular shapes can be used.

The powder, particulates, and shards can be sized by conventional techniques, such as, for example, sieving material through standard screens to the desired sizes. Filtering processes can screen out excessively large and/or excessively fine particles to obtain shards of a desired size. In some embodiments, the particles, powder, or shards have an average size of about 1 micron to about 100 microns. The particles, powder, or shards can have an average size greater than or equal to about 1, 10, 20, 30, 40, 50, 60, 70, 80, or 90 microns;

and/or equal to or less than about 100, 90, 80, 70, 60, 50, 40, 30, 20, or 10 microns, e.g., about 1 to about 20 microns.

The fibers, particulates, powder, and/or shards can be assembled relatively randomly to form radiopaque portion 26, e.g., the fibers may be stacked and cross randomly, to form a network structure. In some embodiments, radiopaque portion 26 can have a packing density percentage of about 30% to about 95%. The packing density percentage can be greater than or equal to about 30%, 35%, 40%, 45%, 50%, 55%, 60%, 70%, 75%, 80%, or 85%; and/or less than or equal to about 95%, 90%, 85%, 80%, 75%, 70%, 65%, 60%, 55%, 50%, 45%, 40%, or 35%. The network structure of radiopaque portion 26 may resemble the microscopic structure of a sponge or of cancellous bone, slightly bonded felt, or three-dimensional layers of netting.

In still other embodiments, radiopaque portion 26 can include mechanical features that help the portion to weaken. For example, radiopaque portion 26 can include indentations or notches that help to provide predictable fracture sites and propagation. Radiopaque portion 26 can include grooves, e.g., circumferential grooves, that segment the radiopaque portion.

The methods described above for forming or modifying radiopaque portion 26 can be used independently or in any combination. For example, the radiopaque material can be annealed and include mechanical features such as grooves. Particles, fibers, and/or shards of radiopaque material can be heat treated, and/or reacted to form a relatively weaker material.

In general, radiopaque portion 26 can be modified at any stage(s) of manufacturing endoprosthesis 20. For example, radiopaque portion 26 can be heat treated and/or embrittled with another material before the portion is incorporated into wire 22. Alternatively or in addition, radiopaque portion 26 can be heat treated and/or embrittled after the radiopaque portion has been incorporated into wire 22, and the wire has been formed into endoprosthesis 20 (described below). In embodiments in which radiopaque portion 26 includes, e.g., particles or fibers, the radiopaque portion can be relatively continuous and intact in wire 22. Subsequently, when wire 22 is formed into endoprosthesis 20 (e.g., by knitting) and/or until the endoprosthesis is placed on a delivery system (e.g., by crimping the endoprosthesis on a balloon), radiopaque portion 26 can weaken, e.g., fracture. Similarly, radiopaque portion 26 that has been heat treated and/or embrittled can be relatively intact and subsequently weakened during formation of endoprosthesis 20 and/or during placement of the endoprosthesis on a delivery system. Mechanical features that help weaken radiopaque portion 26 can be formed on wire 22 and/or on endoprosthesis 20, e.g., during knitting or crimping.

Turning now to outer portion 28, the outer portion can be formed of a biocompatible material that is selected based on the type of endoprosthesis being manufactured. In some embodiments, outer portion 28 is formed of a material suitable for use in a self-expandable endoprosthesis. For example, outer portion 28 can be formed of a continuous solid mass of a relatively elastic biocompatible metal such as a superelastic or pseudo-elastic metal alloy. Examples of superelastic materials include, for example, a Nitinol (e.g., 55% nickel, 45% titanium), silver-cadmium (Ag-Cd), gold-cadmium (Au-Cd), gold-copper-zinc (Au-Cu-Zn), copper-aluminum-nickel (Cu-Al-Ni), copper-gold-zinc (Cu-Au-Zn), copper-zinc/(Cu-Zn), copper-zinc-aluminum (Cu-Zn-Al), copper-zinc-tin (Cu-Zn-Sn), copper-zinc-xenon (Cu-Zn-Xe), iron beryllium (Fe₃Be), iron platinum (Fe₃Pt), indium-thallium (In-Tl), iron-manganese (Fe-Mn), nickel-titanium-vanadium (Ni-Ti-V), iron-nickel-titanium-Cobalt (Fe-Ni-Ti-Co) and copper-tin (Cu-Sn). See, eg., Schetsky, L. McDonald, “Shape Memory Alloys”, Encyclopedia of Chemical Technology (3rd ed.), John Wiley & Sons, 1982, vol. 20. pp. 726-736 for a full discussion of superelastic alloys. Other examples of materials suitable for outer portion 28 include one or more precursors of superelastic alloys, i.e., those alloys that have the same chemical constituents as superelastic alloys, but have not been processed to impart the superelastic property under the conditions of use. Such alloys are further described in PCT application US91/02420.

In other embodiments, outer portion 28 includes materials that can be used for a balloon-expandable endoprosthesis, such as noble metals, such as platinum, gold, and palladium, refractory metals, such as tantalum, tungsten, molybdenum and rhenium, and alloys thereof. Other examples of stent materials include titanium, titanium alloys (e.g., alloys containing noble and/or refractory metals), stainless steels, stainless steels alloyed with noble and/or refractory metals, nickel-based alloys (e.g., those that contained Pt, Au, and/or Ta), iron-based alloys (e.g., those that contained Pt, Au, and/or Ta), and cobalt-based alloys (e.g., those that contained Pt, Au, and/or Ta). Outer portion 28 can include a mixture of two or more materials, in any combination.

Wire 22 can be formed by conventional techniques. For example, wire 22 can be formed by a drawn filled tubing (DFT) process, which can be performed, for example, by Fort Wayne Metals Research (Fort Wayne, Ind.). Generally, the process begins with placing the radiopaque material(s) into a central opening defined by outer portion 28, e.g., a tube, to form a composite wire. Other methods of forming the composite wire include, e.g., coating the radiopaque material with the desired material(s) of outer portion 28 such as by electro- or electroless plating, spraying, e.g., plasma spraying, dipping in molten material, e.g., galvanizing, chemical vapor deposition, and physical vapor deposition. The composite wire can then be put through a series of alternating cold-working, e.g., drawing, and annealing steps that elongate the wire while reducing its diameter to form wire 22. These processing steps can weaken, e.g., fracture, or further weaken radiopaque portion 26. The DFT process is described, for example, in Mayer, U.S. Pat. No. 5,800,511; and J. E. Schaffer, “DFT Biocompatible Wire”, Advanced Materials & Processes, October 2002, pp. 51-54. The composite wire can be in any cross-sectional geometric configurations, such as circular, oval, irregularly or regularly polygonal, e.g., square, triangular, hexagonal, octagonal, or trapezoidal.

The amount of radiopaque portion 26 relative to outer portion 28 can be dependent on a variety of factors, such as, for example, the mass absorption coefficient of the radiopaque material, the thickness of the cross section that is attenuating incident X-rays, the material(s) used for outer portion 28, and the desired radiopacity. A model for forming a composite wire is presented below in Example 2. Generally, in some cases, for a wire having a Nitinol outer portion, the wire includes about 3% by cross-sectional area to about 80% by cross-sectional area of radiopaque material(s). The cross-sectional area can be equal to or greater than about 3%, 5%, 10%, 15%, 20%, 25%, 30%, 35%, 40%, 45%, 50%, 55%, 60%, 65%, 70%, or 75%; and/or equal to or less than about 80%, 75%, 70%, 65%, 60%, 55%, 50%, 45%, 40%, 35%, 30%, 25%, 20%, 15%, 10%, or 5%. Wire 22 can have a diameter about 0.0005 in to about 0.040 in.

After wire 22 is formed, the wire can then be formed into endoprosthesis 20. For example, wires 22 can be wound about a cylindrical form, and the filaments can be locked relative to each other, as described in Mayer, U.S. Pat. No. 5,800,511. Other methods of forming an endoprosthesis include knitting wire 22, e.g., on a circular knitting machine, as described, for example, in Heath, U.S. Pat. No. 5,725,570; Strecker, U.S. Pat. No. 4,922,905; and Andersen, U.S. Pat. No. 5,366,504. Endoprosthesis 20 can be formed from wire 22 by other means such as weaving, crocheting, or forming the wire into a spiral-spring form element. Wire 22 can be incorporated, e.g., by co-knitting, within an endoprosthesis including conventional metal or non-metal materials (e.g. Dacron for an aortic graft) to contribute properties such as strength and/or radiopacity. Wire 22 can be co-knitted with other wires, for example, including pure stainless steel (e.g., 300 series stainless steel), pure shape memory alloys (e.g., Nitinol), or composite materials as described in Heath, U.S. Pat. No. 5,725,570, and Mayer, U.S. Pat. No. 5,800,511.

In general, endoprosthesis 20 can be of any desired shape and size (e.g., coronary stents, aortic stents, peripheral vascular stents, gastrointestinal stents, urology stents, and neurology stents). Depending on the application, stent 10 can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Endoprosthesis 20 can be balloon-expandable, self-expandable, or a combination of both (e.g., U.S. Pat. No. 5,366,504).

Endoprosthesis 20 can be used, e.g., delivered and expanded, according to conventional methods. During use, radiopaque portion 26 does not impede the response or movement of endoprosthesis 20. Suitable catheter systems are described in, for example, Wang U.S. Pat. No. 5,195,969, and Hamlin U.S. Pat. No. 5,270,086. Suitable stents and stent delivery are also exemplified by the Radius® or Symbiot® systems, available from Boston Scientific Scimed, Maple Grove, Minn.

Endoprosthesis 20 can also be a part of a stent-graft. In other embodiments, endoprosthesis 20 can include and/or be attached to a biocompatible, non-porous or semi-porous polymer matrix made of polytetrafluoroethylene (PTFE), expanded PTFE, polyethylene, urethane, or polypropylene. The endoprosthesis can include a releasable therapeutic agent, drug, or a pharmaceutically active compound, such as described in U.S. Pat. No. 5,674,242, U.S. Ser. No. 09/895,415, filed Jul. 2, 2001, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

Still numerous other embodiments are possible.

In certain embodiments, wire for forming endoprosthesis 20 includes more than two layers or portions. Referring to FIG. 3, a wire 50 (as shown, a four-layer structure) includes two radiopaque portions 26 alternating with portions 52. Portions 52 can be made of generally the same material(s) as outer portion 28. Wire 50 can be made, for example, by performing a series of drawn filled tubing processes. Wire 50 can include any number of portions, e.g., three, four, five, six, seven, eight or more.

In some embodiments, wire 22 or 50 includes one or more materials that are visible by magnetic resonance imaging (MRI). For example, the MRI visible material(s) can substitute for the radiopaque material(s) (e.g., in portion 26), be mixed with one or more portions of the radiopaque material(s) (e.g., in wire 50), or form one or more discrete portions of wire 50. The MRI visible material(s) can be formed or modified as described above for radiopaque portion 26. For example, the MRI visible material can be formed to mechanically weaken during use, to be in discontinuous form (e.g., fibers or particles), and/or to include mechanical features that help to weaken the material. Examples of MRI visible materials include non-ferrous metal-alloys containing paramagnetic elements (e.g., dysprosium or gadolinium) such as terbium-dysprosium, dysprosium, and gadolinium; non-ferrous metallic bands coated with an oxide or a carbide layer of dysprosium or gadolinium (e.g., Dy₂O₃ or Gd₂O₃); non-ferrous metals (e.g., copper, silver, platinum, or gold) coated with a layer of superparamagnetic material, such as nanocrystalline Fe₃O₄, CoFe₂O₄, MnFe₂O₄, or MgFe₂O₄; and nanocrystalline particles of the transition metal oxides (e.g., oxides of Fe, Co, Ni).

Alternatively or in addition, the MRI visible material(s) or other low magnetic susceptibility material(s) (such as tantalum, platinum, or gold) can also be used to substitute for a portion of outer portion (e.g., portion 28 or portion(s) 52). For example, in some cases, a material (such as stainless steel) can have sufficiently high magnetic susceptibility to cause signal voids during MRI. By reducing an amount of the material (e.g., stainless steel) with a low magnetic susceptibility material(s), the interaction between the endoprosthesis and an MRI magnetic field is reduced, thereby reducing the magnetic susceptibility void in the area about the endoprosthesis.

The embodiments of wire 22 or 50 described above can be applied to other medical devices. For example, wire 22 or 50 can be used to form filters, such as removable thrombus filters described in Kim et al., U.S. Pat. No. 6,146,404; in intravascular filters such as those described in Daniel et al., U.S. Pat. No. 6,171,327; and in vena cava filters such as those described in Soon et al., U.S. Pat. No. 6,342,062. Wire 22 or 50 can be used to form guidewires, such as a Meier steerable guidewire. Wire 22 or 50 can be used to form vaso-occlusive devices, e.g., coils, used to treat intravascular aneurysms, as described, e.g., in Bashiri et al., U.S. Pat. No. 6,468,266, and Wallace et al., U.S. Pat. No. 6,280,457. Wire 22 or 50 can also be used in surgical instruments, such as forceps, needles, clamps, and scalpels.

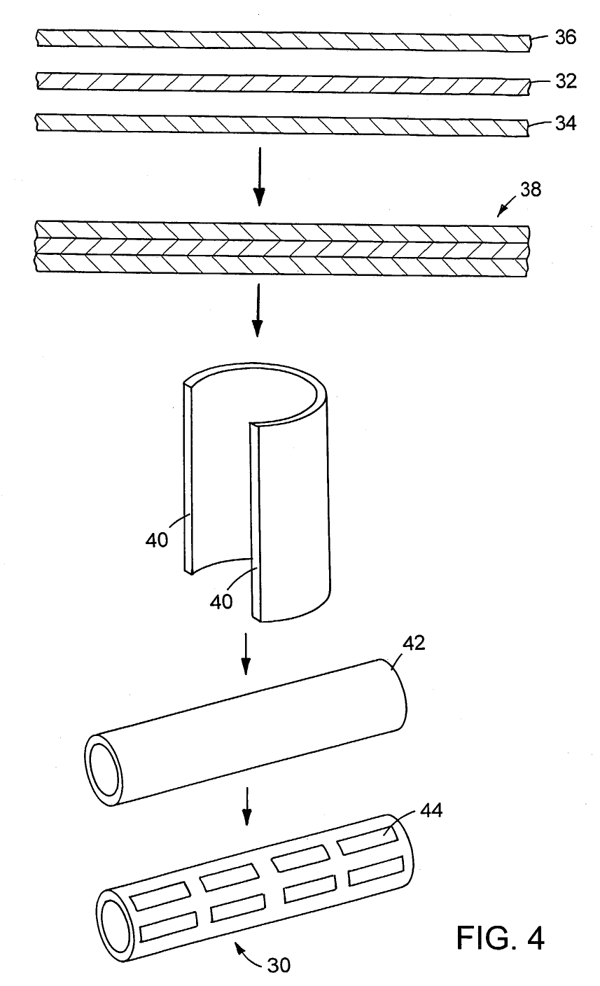

In certain embodiments, an endoprosthesis can be formed from a multilayer structure, e.g., a composite sheet. Referring to FIG. 4, an endoprosthesis 30 (as shown, a tube stent) is formed by laminating a radiopaque layer 32 between an inner layer 34 and an outer layer 36. Radiopaque layer 32 can be generally the same as radiopaque portion 26, e.g., formed relatively weak and/or include selected mechanical features. Inner and outer layers 34 and 36, which can be the same or different, can be generally as described for outer portion 28. Layers 32, 34, and 36 can be laminated together, for example, by heating and pressing, to form a multilayer structure 38. Other methods of forming layers 34 and 36 on radiopaque layer 32 include, for example, electrodeposition, spraying, e.g., plasma spraying, dipping in molten material, e.g., galvanizing, chemical vapor deposition, and physical vapor deposition.

Structure 38 can then be formed into a tube, e.g., by wrapping around a mandrel. Opposing edges 40 of structure 38 can then joined, e.g., by welding, to form a multilayer tube 42. Endoprosthesis 30 can then be formed by forming openings 44 in tube 42, e.g., by laser cutting as described in U.S. Pat. No. 5,780,807. In other embodiments, openings 44 can be formed in structure 38 prior to joining edges 40. Other methods of removing portions of tube 42 or structure 38 can be used, such as mechanical machining (e.g., micro-machining), electrical discharge machining (EDM), and photoetching (e.g., acid photoetching).

In still other embodiments, outer portion 28 or one or more portions 52 include a polymer, such as a shape memory polymer. Suitable polymers include elastomers that are typically crosslinked and/or crystalline and exhibit melt or glass transitions at temperatures that are above body temperature and safe for use in the body, e.g. at about 40 to 50° C. Suitable polymers include polynorbomene, polycaprolactone, polyenes, nylons, polycyclooctene (PCO) and polyvinyl acetate/polyvinylidinefluoride (PVAc/PVDF). A more detailed description of suitable polymers, including shape memory polymers, is available in U.S. S. No. 60/418,023, filed Oct. 11, 2002, and entitled “Endoprosthesis”.

The following examples are illustrative and not intended to be limiting.

### EXAMPLE 1

The following example illustrates a method of making a wire having a Nitinol outer portion and a relatively soft tantalum radiopaque portion.

The recovery stress during a phase transformation of Nitinol has been reported as being on the order of 80 ksi. (See, eg., Material Property Testing of Nitinol Wires, JB Ditman, 1994, American Institute of Aeronautics and Astronautics, Inc.) If, for example, a composite, drawn filled wire of Nitinol/tantalum having a tantalum core diameter of 0.003″ and an outer diameter of 0.006″ were stretched to 8% strain, the Nitinol casing of the wire is expected to exert a recovery stress of 80 ksi while returning to an unstretched length. The recovery load exerted by the Nitinol casing with a cross-sectional area of 2.12×10⁻⁵ square inches is calculated to be 1.7 pounds. An annealed tantalum core is expected to have a yield stress of about 26 ksi or a yield load for the 0.003″ diameter tantalum core wire of 0.2 pounds. (See, eg., Metals Handbook Ninth Edition, Volume 2 Properties and Selection: Nonferrous Alloys and Pure Metals, American Society for Metals, 1979, p. 802 FIG. 98.) The Nitinol is expected to overcome a substantial amount of the resistance to flow from the relative weak core wire until the recovery stress in the Nitinol becomes less than the yield strength of the tantalum.

The composite wire can be formed by performing multiple heat treatments or annealing steps in which tantalum is annealed at relatively high temperatures, e.g., 1200° C. or higher. However, in some embodiments, Nitinol is annealed at about 500° C., and annealing Nitinol at higher temperatures can cause considerable grain growth and adversely affect its mechanical properties. Thus, in some embodiments, the tantalum core wire can be annealed separately and subsequently used as a mandrel, e.g., at a nearly finished size of 0.003″ diameter. A Nitinol tubing can then be drawn down to final dimensions over the tantalum mandrel. The Nitinol tubing can then be annealed and heat set without deleteriously affecting the tantalum because the Nitinol annealing temperatures as substantially lower than the tantalum annealing temperatures. Similar annealing processes can be used to form composite DFT wires having other radiopaque materials, such as gold or platinum.

The annealing processes can also be used to make multilayer tubing. To form a bi-layer tubing, e.g., for stent manufacturing or catheter shafting, the radiopaque core portion can be a tube defining a lumen, rather than a solid wire or tube. To form a tri-layer tubing, two layers of finished or nearly-finished Nitinol, e.g., foil, can be applied, e.g., pressed or rolled, to a layer of soft and annealed radiopaque material. The three-layer structure can be rolled to form a tube and bonded, e.g., by laser welding, to from a tri-layer tubing.

### EXAMPLE 2

The following example illustrates a method for calculating radiopacity for determining the mass and size of radiopaque material in a composite wire.

The mass absorption coefficients (in cm²/g at 50 keV) and densities (in g/cc) of certain materials are listed below in Table 1. The mass absorption coefficient for NiTi is calculated from the rule of mixtures.

Table 1

TABLE 1

| | Ni_{0.5}Ti_{0.5} | Ni | Ta | Ti | Zr | Pt | Au |
|---|---|---|---|---|---|---|---|
| Mass absorption | 1.85 | 2.47 | 5.72 | 1.21 | 6.17 | 6.95 | 7.26 |
| coefficient | | | | | | | |
| Density | 6.5 | 8.9 | 16.7 | 4.5 | 6.5 | 21.5 | 19.3 |

In a composite having 30% by weight platinum (195 g/mole) and 70% by weight Ni_{0.5}Ti_{0.5} (54 g/mole), the atomic percent of Pt in the composite is calculated as follows:

In 100 g of Ni_{0.5}Ti_{0.5}-30% Pt, there is 70 g of NiTi and 30 g of Pt.

(70 g NiTi)(1 mole NiTi/54 g)(6.02×10²³ atoms/mole)=7.80×10²³ atoms NiTi

(30 g Pt)(1 mole Pt/195 g)(6.02×10²³ atoms/mole)=0.93×10²³ atoms Pt

Total=8.73×10²³ atoms in the composite

0.93/8.73=11 atomic percent Pt in the composite

In one example, the radiopacity of a coronary stent (Nitinol outer portion with a platinum core) with a wall thickness of about 0.005 inch is preferably at least about one half that of pure tantalum to be readily visible in fluoroscopy. Pure tantalum coronary stents can appear too bright in fluoroscopic images, and it is believed that about half of that brightness in the image would be sufficient to allow a physician to identify the position of the stent.

The mass absorption coefficient for Ni_{0.5}Ti_{0.5} is estimated by a rule of mixtures calculation to be 1.85, and is reported in the literature to be 5.72 cm²/g for tantalum. Half the mass absorption coefficient of tantalum is 2.86. Using the rule of mixtures for combining mass absorption coefficients, a composite of 20 atomic % platinum and 80 atomic % Ni_{0.5}Ti_{0.5} is about half the mass absorption coefficient of tantalum: 0.20(6.95)+0.80(1.85)=2.87 cm²/g mass absorption coefficient.

Mathematical conversion of atomic percentages to weight percentages for this composite indicates that 53% by weight of Ni_{0.5}Ti_{0.5} and 47% by weight of platinum would have good radiopacity:

For 10²³ atoms total:

(10²³ atoms)(0.20)(195 g/mole)(1 mole/6.02×10²³ atoms)=6.48 g Pt

(10²³ atoms)(0.80)(54 g/mole)(1 mole/6.02×10²³ atoms)=7.18 g Ni_{0.5}Ti_{0.5}

6.48 g Pt/6.48+7.18=0.47 Pt (47 w % Pt)

100−47=53 w % Ni_{0.5}Ti_{0.5}

The total thickness of material presented to incident X-rays in the center of the stent is twice the wall thickness, or in this example, 0.010 inch.

The cross-sectional area of a 0.010 inch wire is (π/4)(0.010)² or 0.000079 square inch.

In a 0.010 inch composite wire having 47% Pt and 53% Ni_{0.5}Ti_{0.5}, the cross-sectional area and diameter of platinum core 26 can be calculated as follows:

mass of Pt+mass of Ni_{0.5}Ti_{0.5}=mass of wire

0.47(mass of wire)+0.53(mass of wire)=mass of wire

mass of Pt=0.47(mass of wire)=(ρ_{Pt})(CSA_{Pt}), where CSA is the cross-sectional area, and ρ is the density

mass of Ni_{0.5}Ti_{0.5}=0.53(mass of wire)=(ρ_{Ni0.5Ti0.5})(CSA_{wire}−CSA_{Pt})

In a one-inch long segment of wire:

(ρ_{Pt})(CSA_{Pt})+(ρ_{Ni0.5Ti0.5})(CSA_{wire}−CSA_{Pt})=[(ρ_{Pt})(CSA_{Pt})]/0.47

Solving for CSA_{Pt}, CSA_{Pt}=0.000016 square inch, and the diameter of the platinum core is 0.0046 inch. Thus, platinum occupies about 20% of the cross-sectional area of a 0.010 inch diameter wire.

All publications, references, applications, and patents referred to herein are incorporated by reference in their entirety.

Other embodiments are within the claims.

What is claimed is:

1. A stent, comprising:
a structure comprising
a first portion comprising a first composition, the first composition fracturing upon expansion of the structure, and
a second portion comprising a second composition less radiopaque than the first composition.

2. The stent of claim 1, wherein the second portion surrounds the first portion.

3. The stent of claim 1, wherein the second composition comprises a shape memory material.

4. The stent of claim 1, wherein the second composition has superelastic characteristics.

5. The stent of claim 1, wherein the second composition comprises a nickel-titanium alloy.

6. The stent of claim 1, wherein the second composition comprises stainless steel.

7. The stent of claim 1, wherein the second composition comprises titanium.

8. The stent of claim 1, wherein the second composition comprises a polymer.

9. The stent of claim 8, wherein the polymer is selected from the group consisting of polynorbomene, polycaprolactone, polyenes, nylons, polycyclooctene (PCO) and polyvinyl acetate/polyvinylidinefluoride.

10. The stent of claim 1, wherein the first composition has a density greater than about 9.9 g/cc.

11. The stent of claim 1, wherein the first composition comprises a material selected from the group consisting of gold, tantalum, palladium, and platinum.

12. The stent of claim 1, wherein the first composition is in the form of a powder.

13. The stent of claim 1, wherein the first composition is in the form of fibers.

14. The stent of claim 1, wherein the structure further comprises a third portion comprising the second composition, and the first portion is between the second and third portions.

15. The stent of claim 1, wherein the structure is in the form of a wire.

16. The stent of claim 1, wherein the structure is a tubular member.

17. The stent of claim 1, in the form of a self-expandable stent.

18. The stent of claim 1, in the form of a balloon-expandable stent.

19. The stent of claim 1, in the form of a stent-graft.

20. The stent of claim 19, wherein the stent-graft comprises a therapeutic agent.

21. A medical device, comprising:
a structure comprising
a first portion comprising a mixture including a radiopaque composition and a second composition, the mixture having a yield strength less than a yield strength of the substantially pure radiopaque composition, and
a second portion comprising a third composition less radiopaque than the mixture.

22. The device of claim 21, wherein the second composition is selected from the group consisting of carbon, nitrogen, hydrogen, calcium, potassium, bismuth, and oxygen.

23. The device of claim 21, wherein the first portion has a yield strength less than about 80 ksi.

24. The device of claim 21, wherein the second portion encapsulates the first portion.

25. The device of claim 21, wherein the third composition comprises a shape memory material.

26. The device of claim 21, wherein the third composition has superelastic characteristics.

27. The device of claim 21, wherein the third composition comprises a nickel-titanium alloy.

28. The device of claim 21, wherein the third composition comprises stainless steel.

29. The device of claim 21, wherein the third composition comprises a shape memory polymer.

30. The device of claim 21, wherein the first composition has a density greater than about 9.9 g/cc.

31. The device of claim 21, wherein the first composition comprises a material selected from the group consisting of gold, tantalum, palladium, and platinum.

32. The device of claim 21, wherein the first composition is in the form of a powder.

33. The device of claim 21, wherein the first composition is in the form of fibers.

34. The device of claim 21, wherein the structure further comprises a third portion comprising the third composition, and the first portion is between the second and third portions.

35. The device of claim 21, wherein the structure is in the form of a wire.

36. The device of claim 21, wherein the structure is a tubular member.

37. The device of claim 21, in the form of a self-expandable stent.

38. The device of claim 21, in the form of a balloon-expandable stent.

39. The device of claim 21, in the form of a stent-graft.

40. The device of claim 39, wherein the stent-graft comprises a therapeutic agent.

41. The device of claim 21, in the form of an intravascular filter.

42. A method of making a medical device, the method comprising:
reducing a yield strength of a radiopaque composition; and
incorporating the radiopaque composition into the medical device.

43. The method of claim 42, wherein reducing the yield strength comprises annealing the radiopaque composition.

44. The method of claim 42, wherein reducing the yield strength comprises reacting the radiopaque composition with a second composition comprising a material selected from the group consisting of carbon, nitrogen, hydrogen, calcium, potassium, bismuth, and oxygen.

45. The method of claim 42, wherein reducing the yield strength comprises removing selected portions of the radiopaque composition.

46. The method of claim 42, wherein the yield strength of radiopaque composition is reduced to less than about 80 ksi.

47. A method of making a medical device, comprising:
forming a structure having a first portion comprising a first composition, and a second portion comprising a second composition less radiopaque than the first composition;
incorporating the structure into the medical device; and
reducing a yield strength of the first composition.

48. The method of claim 47, wherein reducing the yield strength is performed after incorporating the structure into the medical device.

49. The method of claim 47, wherein reducing the yield strength comprises reacting the first composition with a third composition.

50. The method of claim 47, wherein reducing the yield strength comprises heating the first composition.

51. The method of claim 47, wherein the structure is in the form of a wire.

52. The method of claim 47, wherein the structure is in the form of a tube.

53. A method of making a medical device, comprising:
forming a structure having a first portion comprising a first composition, and a second portion comprising a second composition less radiopaque than the first composition; and
incorporating the structure into the medical device, the first composition weakening in response to the incorporating of the structure.

54. The method of claim 53, wherein the medical device includes a stent delivery system.

55. The method of claim 53, further comprising forming the structure into an endoprosthesis.
